SEQUENCE LISTING (9 sequences)

SEQ ID NO: 1 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 2 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 3 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 4 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 5 - protein, 362 aa, Artificial sequence
  Synthetic polypeptide
MKHLWFFLLLVAAPRWVLSGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGGSAGAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 362 aa, Artificial sequence
  Synthetic polypeptide
MKHLWFFLLLVAAPRWVLSGGNCTGCIICSEENGCSTCQQRLFLFIRREGIRRYGKCLHDCPPGYFGIRGQEVNRCKKCGATCESCFSQDFCIRCKRQFYLYKGKCLPTCPPGTLAHQNTRECQGGSAGAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 372 aa, Artificial sequence
  Synthetic polypeptide
MKHLWFFLLLVAAPRWVLSGGNCTGCIICSEENGCSTCQQRLFLFIRREGIRRYGKCLHDCPPGYFGIRGQEVNRCKKCGATCESCFSQDFCIRCKRQFYLYKGKCLPTCPPGTLAHQNTRECQGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGLLQGA

SEQ ID NO: 8 - protein, 361 aa, Artificial sequence
  Synthetic polypeptide
MKHLWFFLLLVAAPRWVLSGGNCTGCIICSEENGCSTCQQRLFLFIRREGIRRYGKCLHDCPPGYFGIRGQEVNRCKKCGATCESCFSQDFCIRCKRQFYLYKGKCLPTCPPGTLAHQNTRECQGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGLLQGA

SEQ ID NO: 9 - protein, 5 aa, Artificial sequence
  Synthetic peptide
LLQGA

(19) United States
(12) Patent Application Publication
(10) Pub. No.: US 2022/0233704 A1
(43) Pub. Date: Jul. 28, 2022

(54) MUTANTS AND DRUG CONJUGATES OF R-SPONDINS AND USE THEREOF

(71) Applicant: THE BOARD OF REGENTS OF THE UNIVERSITY OF TEXAS SYSTEM, Austin, TX (US)

(72) Inventors: Qingyun J. LIU, Houston, TX (US); Kendra S. CARMON, Houston, TX (US); Soohyun PARK, Houston, TX (US); Jie CUI, Houston, TX (US)

(73) Assignee: THE BOARD OF REGENTS OF THE UNIVERSITY OF TEXAS SYSTEM, Austin, TX (US)

(21) Appl. No.: 17/586,258

(22) Filed: Jan. 27, 2022

Related U.S. Application Data
(60) Provisional application No. 63/142,861, filed on Jan. 28, 2021.

(51) Int. Cl. A61K 47/64; A61K 47/65; A61K 51/08; A61K 47/55; A61K 47/60; A61P 35/00

(52) U.S. Cl. CPC A61K 47/6415; A61K 47/65; A61P 35/00; A61K 47/55; A61K 47/60; A61K 51/08

(57) ABSTRACT

Recombinant R-spondin (RSPO) polypeptides are provided, such as RSPO that is fused with Fc and/or comprises a conjugated therapeutic agent. Methods for using such polypeptides, for example in treating cancer are also provided.

### PRIORITY CLAIM

This application claims benefit of priority to U.S. Provisional Application Ser. No. 63/142,861, filed Jan. 28, 2021, the entire contents of which are hereby incorporated by reference.

### INCORPORATION OF SEQUENCE LISTING

The sequence listing that is contained in the file named “UTSHP0374US_ST25.txt”, which is 22 KB (as measured in Microsoft Windows®) and was created on Jan. 26, 2022, is filed herewith by electronic submission and is incorporated by reference herein.

### BACKGROUND

### 1. Field

The present disclosure relates generally to the field of molecular biology, oncology and medicine. More particularly, it concerns recombinant R-spondin polypeptides and methods of using the same for treating cancer.

### 2. Description of Related Art

R-spondins and LGRs form a ligand-receptor system with critical roles in normal development as well as in tumor initiation and progression. LGR4-6 (leucine-rich repeat containing, G protein-coupled receptor 4, 5 and 6) are three related receptors with a large extracellular domain and a seven transmembrane (7TM) domain typical of the rhodopsin family of G protein-coupled receptors. LGR4 is essential for the survival of intestinal stem cells while LGR5 is specifically expressed in adult stem cells of the gastrointestinal system and other tissues (Barker et al., 2013). LGR4-6 bind to the R-spondin group of stem cell factors to potentiate Wnt signaling (Carmon et al., 2011 and de Lau et al., 2011).

R-spondins (RSPOs) are a group of four related secreted proteins with critical roles in organ development and survival of adult stem cells as well as in cancer development (de Lau et al., 2012). The four RSPOs (RSPO1-4) are ˜50% identical to each other in amino acid sequence and constitute an overall similar structure with the N-terminal half containing two cysteine-rich, furin-like domains (Fu1 and Fu2) and the C-terminal half containing a thrombospondin (TSP)-like domain followed by a highly basic region. Aberrant expression of RSPO2 and RSPO3 through gain-of-expression of gene fusions was identified in subsets of colon and other solid tumors as a driving mechanism of oncogenesis (Seshagiri et al., 2012). Overexpression of RSPO3 in lung adenocarcinomas due to transcriptional activation promotes tumor aggressiveness and anti-RSPO3 antibodies are being tested in clinical trials for cancer treatment (Gong et al., 2015 and Chartier et al., 2016).

LGR4-6 are highly upregulated in multiple types of solid tumors. RNA-Seq data of LGR4-6 in TCGA's comprehensive cancer genomics databases showed that LGR4-6 were highly expressed in all cancers of the digestive system, particularly LGR4 and LGRS in colon and liver cancer. Immunohistochemistry (IHC) analysis confirmed that LGR4 and LGRS are highly upregulated in tumors cells with little expression in the stroma (Yi et al., 2013 and Junttila et al., 2015). Furthermore, multiple studies showed that LGR4 and LGRS are enriched in cancer stem cells of the GI system (Shimokawa et al., 2017 and de Sousa e Melo et al., 2017), suggesting that therapeutic targeting of LGR4/5 may lead to killing of cancer stem cells and potentially eradication of cancer.

### SUMMARY

In some embodiments, the present disclosure provides polypeptides comprising a R-spondin (RSPO) domain that comprises a leucine-rich repeat containing, G protein-coupled receptor (LGR) binding sequence and a conjugated therapeutic agent. In some aspects, the polypeptide exhibits reduced Wnt signaling relative to a wild-type RSPO. In some aspects, the polypeptide exhibits reduced RNF43/ZNRF3 binding relative to a wild-type RSPO. In some aspects, the RSPO domain comprises a RSPO Fu1 domain and/or a RSPO TSP domain. In further aspects, the polypeptide comprises a mutation in the Fu1 domain relative to a wildtype RSPO. In still further aspects, the polypeptide comprises an amino acid substitution at a position corresponding to position Arg60, Gln65, and/or Gly67 of the RSPO domain (as numbered in an RSPO consensus sequence of FIG. 1). In yet further aspects, the polypeptide comprises an amino acid substitution at a position corresponding to position Gln65 of the RSPO domain (as numbered in an RSPO consensus sequence of FIG. 1). In still further aspects, the polypeptide comprises an amino acid substitution at a position corresponding to position Gln65 to an Arg (as numbered in an RSPO consensus sequence of FIG. 1).

In some aspects, the RSPO domain comprises at least 85% identity to SEQ ID NOs: 1-4. In further aspects, the RSPO domain comprises at least 90% identity to SEQ ID NOs: 1-4. In still further aspects, the RSPO domain comprises at least 90% identity to SEQ ID NO: 4. In other aspects, the RSPO domain comprises at least 90% identity to SEQ ID NOs: 1-4 and comprises an amino acid substitution at a position corresponding to position Arg60, Gln65, and/or Gly67 of the RSPO domain (as numbered in an RSPO consensus sequence of FIG. 1). In still other aspects, the RSPO domain comprises at least 90% identity to SEQ ID NO: 4 and comprises an amino acid substitution at a position corresponding to position Gln65 of the RSPO domain (as numbered in an RSPO consensus sequence of FIG. 1).). In still other aspects, the RSPO domain comprises at least 95% identity to SEQ ID NO: 4 and comprises an amino acid substitution at a position corresponding to position Gln65 of the RSPO domain (as numbered in an RSPO consensus sequence of FIG. 1).

In some aspects, the polypeptide further comprises a fused Ig Fc domain. In further aspects, the Ig Fc domain is an IgG Fc domain. In still further aspects, the IgG Fc domain is an IgG1 Fc domain. In some aspects, the Ig Fc domain position C-terminal relative to the RSPO domain. In further aspects, the Ig Fc domain is separated from to the RSPO domain by a linker peptide. In still further aspects, the linker peptide is an IgG hinge sequence. In yet further aspects, the linker peptide is an IgG1 hinge sequence. In some aspects, the conjugated therapeutic agent is conjugated to the Fc domain.

In some aspects, the polypeptides further comprise an additional fused amino acid sequence and wherein the conjugated therapeutic agent is conjugated to the additional fused amino acid sequence. In further aspects, the additional fused amino acid sequence comprises a “Q” position that serves as the recipient group for a transglutaminase. In still further aspects, the additional fused amino acid sequence comprises the sequence LLQGA (SEQ ID NO: 9). In some aspects, the additional fused amino acid sequence is position at the C-terminus of the polypeptide. In some aspects, the conjugated therapeutic agent is conjugated via a PEG linker. In some aspects, the conjugated therapeutic agent is conjugated via a PEG-VC-PAB linker. In some aspects, the conjugated therapeutic agent is conjugated by a transglutaminase reaction.

In some aspects, the polypeptide is at least 85% identical to SEQ ID NOs: 5-8. In some aspects, the conjugated therapeutic agent is cytotoxic agent. In some aspects, the conjugated therapeutic agent is cytotoxic agent. In some aspects, the conjugated therapeutic agent is chemotherapeutic agent. In some aspects, the conjugated therapeutic agent comprises a radioactive isotope. In some aspects, the conjugated therapeutic agent comprises a MMAE (monomethyl-auristatin) or DMSA. In further aspects, the conjugated therapeutic agent is conjugated by a transglutaminase reaction with NH4-PEG4-VC-PAB-MMAE or NH2-PEG4-vc-PAB-DMEA-(PEG2)-Duocarymicin SA.

In other embodiments, the present disclosure provides methods of treating a subject comprising administering an effective amount of a polypeptide of the present disclosure. In some aspects, the subject has cancer. In further aspects, the subject has a solid tumor. In some aspects, the cancer is colon or liver cancer. In some aspects, the methods further comprise administering at least a second anticancer therapy to the subject. In further aspects, the second anticancer therapy is a surgical therapy, chemotherapy, radiation therapy, cryotherapy, hormonal therapy, immunotherapy or cytokine therapy.

In still other embodiments, the present disclosure provides polypeptides comprising a R-spondin (RSPO) domain that comprises a leucine-rich repeat containing, G protein-coupled receptor (LGR) binding sequence and fused Ig Fc domain. In some aspects, the polypeptide exhibits reduced Wnt signaling relative to a wild-type RSPO. In some aspects, the polypeptide exhibits reduced RNF43/ZNRF3 binding relative to a wild-type RSPO. In some aspects, the RSPO domain comprises a RSPO Fu1 domain and/or a RSPO TSP domain. In further aspects, the polypeptide comprises a mutation in the Fu1 domain relative to a wildtype RSPO. In still further aspects, the polypeptide comprises an amino acid substitution at a position corresponding to position Arg60, Gln65, and/or Gly67 of the RSPO domain (as numbered in an RSPO consensus sequence of FIG. 1). In yet further aspects, the polypeptide comprises an amino acid substitution at a position corresponding to position Gln65 of the RSPO domain (as numbered in an RSPO consensus sequence of FIG. 1). In further aspects, the polypeptide comprises an amino acid substitution at a position corresponding to position Gln65 to an Arg (as numbered in an RSPO consensus sequence of FIG. 1).

In some aspects, the RSPO domain comprises at least 85% identity to SEQ ID NOs: 1-4. In further aspects, the RSPO domain comprises at least 90% identity to SEQ ID NOs: 1-4. In still further aspects, the RSPO domain comprises at least 90% identity to SEQ ID NO: 4. In some aspects, the RSPO domain comprises at least 90% identity to SEQ ID NOs: 1-4 and comprises an amino acid substitution at a position corresponding to position Arg60, Gln65, and/or Gly67 of the RSPO domain (as numbered in an RSPO consensus sequence of FIG. 1). In some aspects, the RSPO domain comprises at least 90% identity to SEQ ID NO: 4 and comprises an amino acid substitution at a position corresponding to position Gln65 of the RSPO domain (as numbered in an RSPO consensus sequence of FIG. 1). In some aspects, the RSPO domain comprises at least 95% identity to SEQ ID NO: 4 and comprises an amino acid substitution at a position corresponding to position Gln65 of the RSPO domain (as numbered in an RSPO consensus sequence of FIG. 1).

In some aspects, the Ig Fc domain is an IgG Fc domain. In further aspects, the IgG Fc domain is an IgG1 Fc domain. In some aspects, the Ig Fc domain position C-terminal relative to the RSPO domain. In further aspects, the Ig Fc domain is separated from to the RSPO domain by a linker peptide. In still further aspects, the linker peptide is an IgG hinge sequence. In yet further aspects, the linker peptide is an IgG1 hinge sequence. In some aspects, the polypeptide further comprises a conjugated therapeutic agent. In further aspects, the conjugated therapeutic agent is conjugated to the Fc domain.

In some aspects, the polypeptides further comprise an additional fused amino acid sequence and wherein the conjugated therapeutic agent is conjugated to the additional fused amino acid sequence. In further aspects, the additional fused amino acid sequence comprises a “Q” position that serves as the recipient group for a transglutaminase. In still further aspects, the additional fused amino acid sequence comprises the sequence LLQGA (SEQ ID NO: 9). In some aspects, the additional fused amino acid sequence is position at the C-terminus of the polypeptide. In some aspects, the conjugated therapeutic agent is conjugated via a PEG linker. In some aspects, the conjugated therapeutic agent is conjugated via a PEG-VC-PAB linker. In some aspects, the conjugated therapeutic agent is conjugated by a transglutaminase reaction. In some aspects, the polypeptide is at least 85% identical to SEQ ID NOs: 5-8. In some aspects, the conjugated therapeutic agent is cytotoxic agent. In some aspects, the conjugated therapeutic agent is cytotoxic agent. In some aspects, the conjugated therapeutic agent is chemotherapeutic agent. In some aspects, the conjugated therapeutic agent comprises a radioactive isotope. In some aspects, the conjugated therapeutic agent comprises a MMAE (monomethyl-auristatin) or DMSA. In further aspects, the conjugated therapeutic agent is conjugated by a transglutaminase reaction with NH4-PEG4-VC-PAB-MMAE or NH2-PEG4-vc-PAB-DMEA-(PEG2)-Duocarymicin SA.

In yet other embodiments, the present disclosure provides methods of treating a subject comprising administering an effective amount of a polypeptide of the present disclosure. In some aspects, the subject has cancer. In further aspects, the subject has a solid tumor. In some aspects, the cancer is colon or liver cancer. In some aspects, the methods further comprise administering at least a second anticancer therapy to the subject. In further aspects, the second anticancer therapy is a surgical therapy, chemotherapy, radiation therapy, cryotherapy, hormonal therapy, immunotherapy or cytokine therapy.

In other embodiments, the present disclosure provides nucleic acid molecules encoding a polypeptide of the present disclosure.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Alignment of RSPO sequences and the residues critical for binding to RNF43/ZNRF1. The protein sequences of human RSPO1 (SEQ ID NO: 1); RSPO2 (SEQ ID NO: 2); RSPO3 (SEQ ID NO: 3); and RSPO4 (SEQ ID NO: 4) are aligned and the three domains, Fu1, Fu2, and TSP are marked by various lines. The three amino acid residues in the Fu1 domain, corresponding to Arg60, Gln65, and Gly67 of RSPO4, which are essential for binding to RNF43/ZNRF3, are boxed. *denotes absolutely conserved residues.

FIG. 2: A schematic diagram illustrating the interactions of R spondin (RSPO) with LGR and RNF43/ZNRF3 in regulating Wnt signaling. RNF43 and ZNRF3 are two related E3 ubiquitin ligases that ubiquitinate Wnt receptor for degradation. On the left side, the furin (Fu1) domain of RSPO wild-type (WT) binds to the extracellular domain of RNF43/ZNRF while the Fu2 domain is anchored to LGR4/5/6. This leads to inhibition of RNF43/ZNRF3 activity and increase in Wnt receptor level, and therefore enhancement of Wnt signaling. On the right side, mutations of RSPO1-Fu1 residues involved in binding to RNF43/ZNRF results in loss of binding to RNF43/ZNRF3 which will ubiquitinate Wnt receptor for degradation, and therefor decrease Wnt signaling. Meanwhile, the RSPO-Fu 1 mutants remain capable of binding to LGR with high affinity and are co-internalized with the receptors.

FIGS. 3A-3C:Mutations in Fu1 domain of RSPO4 maintains binding activity to LGR4 but lost Wnt-potentiating activity. (FIG. 3A) Saturation binding analysis of RSPO4-furin Fc fusion proteins R403 and R405 to HEK293 cells overexpressing LGR4. (FIG. 3B) TOPFlash Wnt/beta-catenin signaling activity of R403 and R405 in HEK293-STF cells. (FIG. 3C) TOPFlash Wnt/beta-catenin signaling activity of RSPO1 (100 ng/mL) in the presence of increasing concentrations of R405.

FIG. 4: Binding of RSPO4 furin mutant to the colon cancer cell line LoVo cells without and with knockout. R405 was incubated with the cells at 37° C. for 1 hr, followed by washing, fixing, and permeabilization, and then detected with Alexa488-labeled anti-Human IgG1, and visualized by confocal microscopy.

FIGS. 5A-5B:Cell killing activity of R4Fu-Fc proteins in the presence of drug conjugated anti-human Fab fragment. (FIG. 5A) Cell viability of HEK293T cells with or without over-expressing LGR4 in the presence of increasing concentrations of R403 and R405 plus a fixed concentration (2 μg/mL) of MMAE-conjugated anti-human Fc Fab. (FIG. 5B) Cell viability of cancer cell lines LoVo and Ovcar3 cells in the presence of increasing concentrations of R403 and R405 plus a fixed concentration (2 μg/mL) of MMAE-conjugated anti-human Fc Fab.

FIGS. 6A-6B:Site-specific conjugation of cytotoxin to R427. (FIG. 6A) A schematic diagram of the R4Fc-Q65R-Fc mutant with an LLQGA (SEQ ID NO: 9) tag at the C-terminus. The Q (Gln) residue of the tag is the recipient of PEG4-VC-PAB-MMAE catalyzed by microbial glutamine transferase. (FIG. 6B) Coomassie blue staining of R427 before and after conjugation with PEG4-VC-PAB-MMAE.

FIGS. 7A-7C:Cell killing activity of R427-MMAE and R432-DMSA. (FIG. 7A) Cell viability of parental HEK293T cells, HEK293T cells overexpressing LGR4 or LGR5 (HEK293T-LGR4 or -LGR5) with increasing concentrations of R427-MMAE. (FIG. 7B) Cell viability of three cancer cell lines, LoVo, Hep3B, and HepG2 with increasing concentration of the PDC. (FIG. 7C) Cell viability of HepG2 and LoVo cells in increasing concentrations of R432-DMSA.

FIG. 8: Anti-tumor effect of R427-MMAE and R432-DMSA in xenograft models in vivo. LoVo cells were implanted into the dorsal flanks of athymic nude mice. When tumors reached ˜150 mm³ in size, the animals were randomized and injected with vehicle (PBS), R427 (unconjugated), R427-MMAE, or R432-DMSA at 5 mg/kg by intraperitoneal injection every other day for 18 days.

### DESCRIPTION OF ILLUSTRATIVE EMBODIMENTS

### I. The Present Embodiments

In certain embodiments, there is provided a recombinant RSPO polypeptide that is conjugated to a therapeutic agent and/or fused to an Ig Fc domain. Such recombinant polypeptide may be used for the treatment of cancers such as solid tumors.

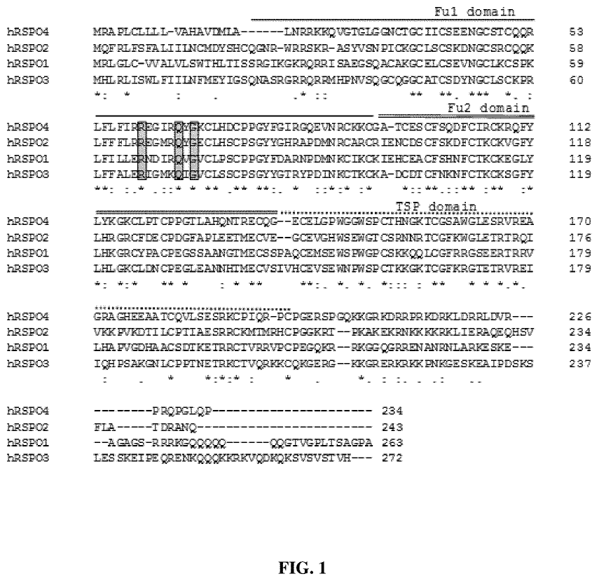

RSPO4 fused to IgG1-Fc
SEQ ID NO: 5
>R403 protein sequence
MKHLWFFLLLVAAPRWVLSGGNCTGCIICSEENGCSTCQQRLFLFIRREG
IRQYGKCLHDCPPGYFGIRGQEVNRCKKCGATCESCFSQDFCIRCKRQFY
LYKGKCLPTCPPGTLAHQNTRECQGGSAGAEPKSCDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNH
YTQKSLSLSPGK*
RSPO4 fused to IgG1-Fc, Gln65 (based on the
consensus numbering, see FIG. 1) substituted for
Arg (“Q65R”)
SEQ ID NO: 6
>R405 protein sequence:
MKHLWFFLLLVAAPRWVLSGGNCTGCIICSEENGCSTCQQRLFLFIRREG
IRRYGKCLHDCPPGYFGIRGQEVNRCKKCGATCESCFSQDFCIRCKRQFY
LYKGKCLPTCPPGTLAHQNTRECQGGSAGAEPKSCDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNH
YTQKSLSLSPGK*
RSPO4-Fu-Q65R-Fc with an LLQGA (SEQ ID NO: 9) tag
at the C-terminus
SEQ ID NO: 7
>R427 Protein sequence:
MKHLWFFLLLVAAPRWVLSGGNCTGCIICSEENGCSTCQQRLFLFIRREG
IRRYGKCLHDCPPGYFGIRGQEVNRCKKCGATCESCFSQDFCIRCKRQFY
LYKGKCLPTCPPGTLAHQNTRECQGSGGGGSGGGGSGGGGSDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGLLQGA*
SEQ ID NO: 8
>R432 protein sequence
MKHLWFFLLLVAAPRWVLSGGNCTGCIICSEENGCSTCQQRLFLFIRREG
IRRYGKCLHDCPPGYFGIRGQEVNRCKKCGATCESCFSQDFCIRCKRQFY
LYKGKCLPTCPPGTLAHQNTRECQGEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGLLQGA*

### II. RSPO Proteins

Following the discovery of hRSPO3, the first member of the Rspo gene family, other Rspo genes from different species were subsequently discovered. RSPO (roof plate-specific spondin) gene was adopted from the mouse Rspo1 gene that is expressed in the roof plate of the neural tube of developing embryos. In mammals, the RSPO protein family consists of four members, namely RSPO1 through RSPO4 sharing 40-60% amino acid sequence identity with significant homologies within human and mouse members. RSPO2 was identified by a functional screening study in Xenopus as a gene encoding a novel canonical WNT/β-catenin signaling activator and subsequent work confirmed that other RSPO proteins from different species have a similar capacity to activate WNT/β-catenin signaling.

For example, RSPRO1 is a secreted protein that in humans is encoded by the Rspo1 gene, found on chromosome 1. In humans, it interacts with WNT4 in the process of female sex development. Loss of function can cause female to male sex reversal. Furthermore, it promotes canonical WNT/β catenin signaling. The protein has two cysteine-rich, furin-like domains and one thrombospondin type 1 domain.

RSPO1 is required for the early development of gonads, regardless of sex. It has been found in mice only eleven days after fertilization. To induce cell proliferation, it acts synergistically with WNT4. They help stabilize β-catenin, which activates downstream targets. If both are deficient in XY mice, there is less expression of SRY and a reduction in the amount of SOX9. Moreover, defects in vascularization are found. These occurrences result in testicular hypoplasia. Male to female sex reversal, however, does not occur because Leydig cells remain normal. They are maintained by steroidogenic cells, now unrepressed.

RSPO1 is necessary in female sex development. It augments the WNT/β catenin pathway to oppose male sex development. In critical gonadal stages, between six and nine weeks after fertilization, the ovaries upregulate it while the testes downregulate it.

Oral mucosa has been identified as a target tissue for RSPO1. When administered to normal mice, it causes nuclear translocation of β-catenin to this region. Modulation of the WNT/β catenin pathway occurs through the relief of Dkkl inhibition. This occurrence results in increased basal cellularity, thickened mucosa, and elevated epithelial cell proliferation in the tongue. RSPO1 can therefore potentially aid in the treatment of mucositis, which is characterized by inflammation of the oral cavity. This unfortunate condition often accompanies chemotherapy and radiation in cancer patients with head and neck tumors. RSPO1 has also been shown to promote gastrointestinal epithelial cell proliferation in mice.

RSPO2 is a secreted protein that in humans that synergizes with canonical WNT to activate beta-catenin. RSPO2 has been proposed to regulate craniofacial patterning and morphogenesis within pharyngeal arch 1 through ectoderm-mesenchyme signaling via the endothelin-D1x5/6 pathway. In dogs, a variant on the Rspo2 gene is associated moustache and eyebrow thickness. In humans, recessive mutations in RSPO2 abrogate limb and lung development. Bruno Reversade and colleagues have reported in 2018 that loss of RSPO2 results in a syndrome of Tetra-amelia with lung agenesis.

RSPO3 is a protein that in humans is encoded by the RSPO3 gene. This gene encodes a member of the thrombospondin type 1 repeat supergene family. In addition, the protein contains a furin-like cysteine-rich region. Furin-like repeat domains have been found in a variety of eukaryotic proteins involved in the mechanism of signal transduction by receptor tyrosine kinases. During embryonic development, RSPO3 is expressed in the tail bud and the posterior presomitic mesoderm of the embryo. In tissue engineering, R-spondin 3 has been used to differentiate pluripotent stem cells into paraxial mesoderm progenitors.

The RSPO4 gene encodes a member of the R-spondin family sharing a common domain organization consisting of a signal peptide, cysteine-rich/furin-like domain, thrombospondin domain and a C-terminal basic region. The encoded protein may be involved in activation of Wnt/beta-catenin signaling pathways. Mutations in this gene are associated with anonychia congenital. Alternate splicing results in multiple transcript variants.

### III. RSPO Nucleic Acids

Various genetic constructs are available that contain the RSPO agents described above, and these may be introduced into vectors for expression. Nucleic acids according to the present disclosure that encode prodrug molecules may be optionally linked to other protein sequences. Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

A. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present disclosure.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

B. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

C. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

D. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

E. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

F. Origins of Replication

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

G. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

H. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher's disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

I. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection. In certain embodiments, a nucleic acid may be delivered to an organelle, a cell, a tissue or an organism via one or more injections (i.e., a needle injection), such as, for example, either subcutaneously, intradermally, intramuscularly, intravenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation. In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human K-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate. In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran. In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading. Additional embodiments of the present disclosure include the introduction of a nucleic acid by direct sonic loading. LTK⁻fibroblasts have been transfected with the thymidine kinase gene by sonication loading (Fechheimer et al., 1987).

Liposome-Mediated Transfection. In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection. Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

J. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene's Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. pGEM-T Easy vectors, pCon Vectors™, Lonza pConIgG1 or pConK2 plasmid vectors, and 293 Freestyle cells or Lonza CHO cells are also useful for expression of the disclosed prodrug constructs.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary (CHO), W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr that confers resistance to; gpt, which confers resistance to mycophenolic acid; neo, that confers resistance to the aminoglycoside G418; and hygro, which confers resistance to hygromycin.

K. Purification

In certain embodiments, the interferon prodrugs of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an RSPO peptide of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide. Commonly, antibodies are fractionated utilizing agents (i.e., protein A) that bind the Fc portion of the antibody. Where the interferon prodrug contains such a domain, this approach could be used.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### IV. Cancer Therapies

A. RSPO4 Conjugates

RSPO4 of the present disclosure may be linked to at least one agent to form an RSPO4 conjugate. In order to increase the efficacy of RSPO4 conjugate molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which may be attached to RSPO4 include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which may be conjugated to RSPO4 include enzymes, radiolabels, haptens, fluorescent labels, phosphorescent molecules, chemiluminescent molecules, chromophores, photoaffinity molecules, colored particles or ligands, such as biotin.

RSPO4-Fc conjugates may also be used as diagnostic agents, for example, by coupling imaging agents for use in vivo diagnostic protocols. The imaging moieties used can be paramagnetic ions, radioactive isotopes, fluorochromes, NMR-detectable substances, and X-ray imaging agents. In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

Radioactively labeled RSPO4 conjugates of the present disclosure may be produced according to well-known methods in the art. For instance, RSPO4 conjugates can be iodinated by contact with sodium and/or potassium iodide and a chemical oxidizing agent such as sodium hypochlorite, or an enzymatic oxidizing agent, such as lactoperoxidase. RSPO4-Fc conjugates according to the disclosure may be labeled with technetium 99 by ligand exchange 25 process, for example, by reducing pertechnate with stannous solution, chelating the reduced technetium onto a Sephadex column and applying the peptide to this column. Alternatively, direct labeling techniques may be used, e.g., by incubating pertechnate, a reducing agent such as SNCh, a buffer solution such as sodium-potassium phthalate solution, and the peptide. Intermediary functional groups which are often used to bind radioisotopes which exist as metallic ions to peptide are diethylenetriaminepentaacetic acid (DTPA) or ethylene diaminetetracetic acid (EDTA).

B. Pharmaceutical Compositions and Methods of Treatment

The present disclosure provides pharmaceutical compositions comprising an R-spondin (RSPO) domain that comprises a leucine-rich repeat containing, G protein-coupled receptor (LGR) binding sequence and a conjugated therapeutic agent. Such compositions comprise a prophylactically or therapeutically effective amount of the peptide/polypeptide and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the RSPO agent, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

RSPO4 conjugates of the present disclosure, as described herein, can be formulated for parenteral administration, e.g., formulated for injection via the intradermal, intravenous, intramuscular, subcutaneous, intra-tumoral or even intraperitoneal routes. The RSPO4 conjugates could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Combination Therapy

It may also be desirable to provide combination treatments using agents of the present disclosure in conjunction with additional anti-cancer therapies. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the RSPO agent and the other includes the other agent.

Alternatively, the agent of the present disclosure may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several 10 days (2, 3, 4, 5, 6 or 7) to several weeks (1, 2, 3, 4, 5, 6, 7 or 8) lapse between the respective administrations.

It also is conceivable that more than one administration of either the RSPO agent or the other therapy will be desired. Various combinations may be employed, where the agent of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. To kill cells, inhibit cell growth, inhibit metastasis, inhibit angiogenesis or otherwise reverse or reduce the malignant phenotype of tumor cells, using the methods and compositions of the present invention, one may contact a target cell or site with an RSPO agent and at least one other therapy. These therapies would be provided in a combined amount effective to kill or inhibit proliferation of cancer cells. This process may involve contacting the cells/site/subject with the agents/therapies at the same time.

Particular agents contemplated for combination therapy with RSPO conjugates of the present disclosure include chemotherapy and hematopoietic stem cell transplantation. Chemotherapy may include cytarabine (ara-C) and an anthracycline (most often daunorubicin), high-dose cytarabine alone, all-trans-retinoic acid (ATRA) in addition to induction chemotherapy, usually an anthracycline, histamine dihydrochloride (Ceplene) and interleukin 2 (Proleukin) after the completion of consolidation therapy, gemtuzumab ozogamicin (Mylotarg) for patients aged more than 60 years with relapsed AML who are not candidates for high-dose chemotherapy, clofarabine, as well as targeted therapies, such as kinase inhibitors, farnesyl transferase inhibitors, decitabine, and inhibitors of MDR1 (multidrug-resistance protein), or arsenic trioxide or relapsed acute promyelocytic leukemia (APL).

In certain embodiments, the agents for combination therapy are one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a checkpoint inhibitor, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRP 1α antibody or fusions protein, a CD70 antibody, and CLL1 antibody, a CD123 antibody, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Identification of RSPO4 Mutants that Retain Binding to LGRs without Potentiating Wnt Signaling Activity

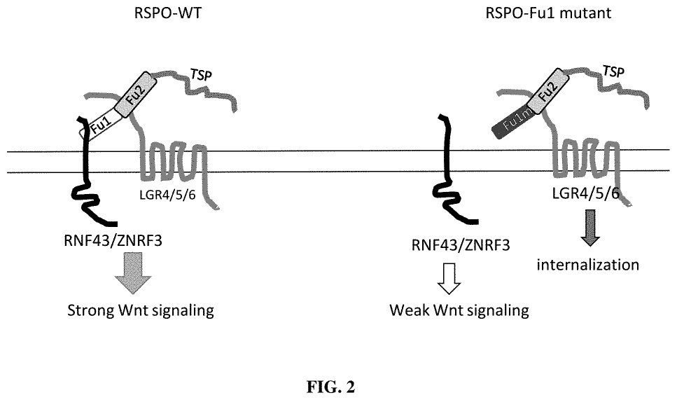

All four R-spondins consist of two furin (Fu)-like domains (Fu1 and Fu2) and thrombospondin type I repeat-like domain (TSP) as illustrated in FIG. 1. The Fu1 domain binds to the E3 ligases RNF43 and ZNRF43 whereas the Fu2 domain binds to LGR4/5/6 (FIG. 2). In particular, three amino acid residues in the Fu1 domain, corresponding to Arg60, Gln65, and Gly67 of RSPO4 (FIG. 1), are essential for binding to RNF43/ZNRF3 based on structure analysis of RSPO-RNF43/ZNRF3 co-crystals. The Fu2 domain of RSPOs bind to LGRs (FIG. 2). Importantly, binding to RNF43/ZNRF3 is essential for RSPOs to potentiate Wnt signaling whereas binding to LGRs further increase activity of the RSPOs.

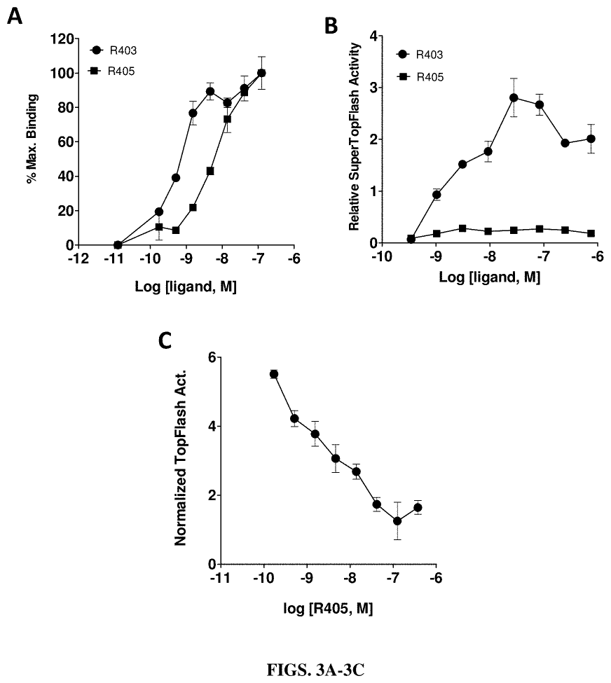

An expression construct was generated that links the Fu1 domain of RSPO4 to human IgG1-Fc to create a fusion protein, often called a peptibody (fusion of a peptide to antibody) using standard molecular cloning methods as described (Park et al., 2018, which is incorporated herein by reference in its entirety). The predicted protein sequence of this peptibody, designated R403 is described in SEQ ID NO: 5. R403 was expressed and purified the protein by protein A/G affinity chromatography following expression in HEK293F cells. It showed high affinity binding to LGR4 (FIG. 3A) and potent activity in potentiating Wnt/β-catenin signaling as measured by the TOPFlash assay (FIG. 3B). A mutant of R403 with Gln65 changed to Arg was generated, as described in SEQ ID NO: 6. The mutant protein, designated R405, was expressed and purified as R403. R405 also showed high affinity binding to LGR4 (FIG. 3A), but completely lost activity in potentiating Wnt signaling (FIG. 3B). Since R405 retained binding to LGR4, we tested if it could antagonize activity of RSPO1 since RSPO1 requires LGR4 to enhance Wnt signaling. As shown in FIG. 3C, R405 was able to inhibit RSPO1 activity in a dose-dependent fashion.

### Example 2—RSPO4-Fu Peptibody Binds to LGR4/5 in Cancer Cells and is Rapidly Internalized

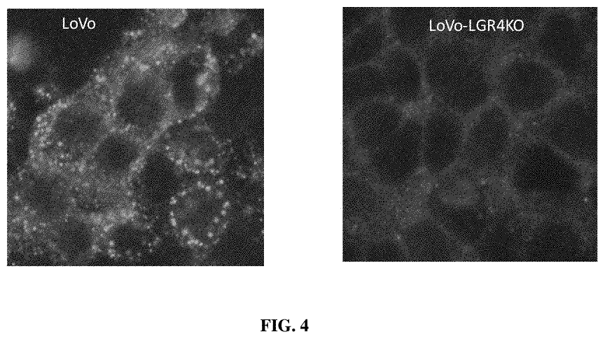

To test if RSPO4-Fu-Fc mutant could bind to LGRs expressed endogenously in cancer cell lines, R405 was incubated at 37° C. with live LoVo cells, a colon cancer cell line that express LGR4 and LGR5 endogenously. Strong binding signal was detected in vesicles, indicating internalization and endocytosis (FIG. 4, left panel). In contrast, no signal was detected in LoVo cells with knockout of LGR4 which also lost expression to LGR5 (FIG. 4, right panel).

### Example 3—RSPO4-Fu Peptibody Effectively Inhibit the Growth of Cancer Cells Expressing LGRs in the Presence of Drug-Conjugated Secondary Antibody

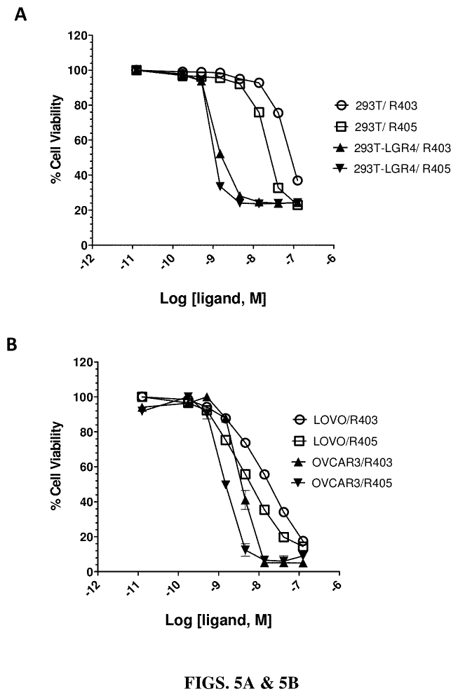

To test if RSPO4-Fu-Fc fusion proteins could serve as carriers of cytotoxic drugs, a secondary antibody-drug conjugate assay was used in which a cytotoxin-conjugated anti-Fc antibody or Fab is added together with the Fc-containing proteins onto cells that may bind to the Fc-containing protein. In the presence of MMAE (monomethyl-auristatin, a potent cytotoxin)-conjugated Fab of anti-human Fc IgG, both R403 and R405 showed much more potent cytotoxic effect on HEK293T cells overexpressing LGR4 than on parental HEK293T cells (FIG. 5A). In two cancer cell lines that express LGR4, both R403 and R405 also showed potent cytotoxic effect (FIG. 5B).

### Example 4—Site-Specific Conjugation of MMAE to RSPO4-Fu Peptibody

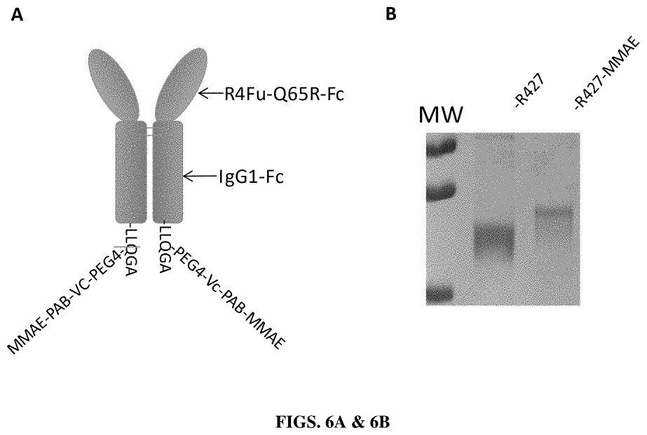

To test the potential of RSPO4-Fu peptibody in carrying out drug delivery, RSPO4-Fu-Q65R-Fc with an LLQGA (SEQ ID NO: 9) tag at the C-terminus of the protein was expressed and purified for site-specific conjugation of cytotoxin (FIG. 6A). The protein sequence of this peptibody, designated R427, is described in SEQ ID NO: 6. The Q (Gln) residue in the LLQGA (SEQ ID NO: 9) tag serves as the recipient group for bacterial transglutaminase (Panowski et al., 2014). The purified peptibody was mixed with the linker-drug NH4-PEG4-VC-PAB-MMAE (MW=˜1500) and bacterial transglutaminase as described (Strop et al., 2013). After incubating overnight at room temperature, the reaction mixture was purified by protein A chromatorgrapy and analyzed by SDA-PAGE. As shown in FIG. 6B, the molecular weight of the protein increased by ˜1500, suggesting that the protein was successfully conjugated to the linker-drug. The resulting peptibody-drug conjugate (PDC) is designated R427-MMAE.

### Example 5—Cytotoxicity of Drug Conjugates of RSPO-Fu-065R-Fc in Cancer Cell Lines

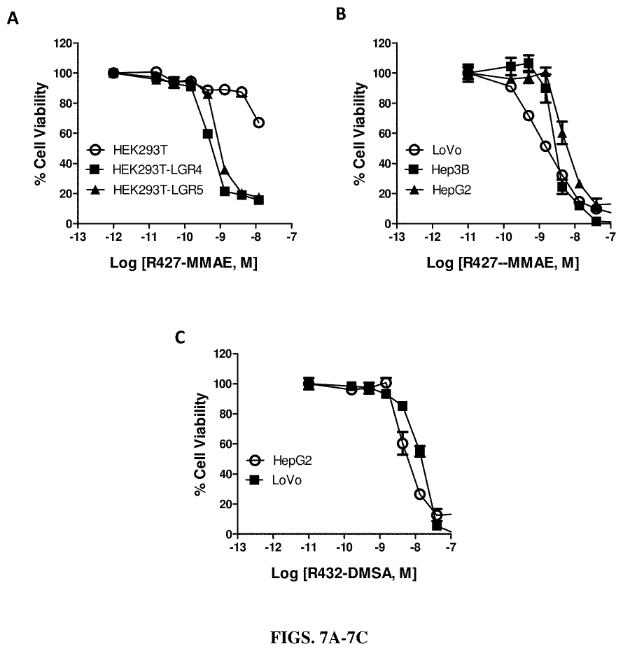

The activity of R427-MMAE in HEK293T cells expressing LGR4 or LGRS was tested. As shown in FIG. 7A, the PDC showed high potent cytotoxic effect on HEK293T-LGR4 or—LGRS cells when compared to parental HEK293T cells. In cancer cell lines that express both LGR4 and LGRS (LoVo and HepG3) or LGR4 alone (Hep3B), the PDC also showed potent cytotoxic effect (IC50<10 nM) (FIG. 7B). Another peptibody of RSPO4-Fu-Q65R-Fc with a different linker was also generated, as detailed in SEQ ID NO; 8. The protein, designated R432, was conjugated with a duocarymicin derivative using the linker-payload NH2-PEG4-vc-PAB-DMEA-(PEG2)-Duocarymicin SA and transglutaminase. The resulting PDC, designated R432-DMSA, showed potency cytotoxic effect in HepG2 and LoVo cells (FIG. 7C).

### Example 6—Anti-Tumor Activity of Drug Conjugates of RSPO4-Fu-065R-Fc In Vivo

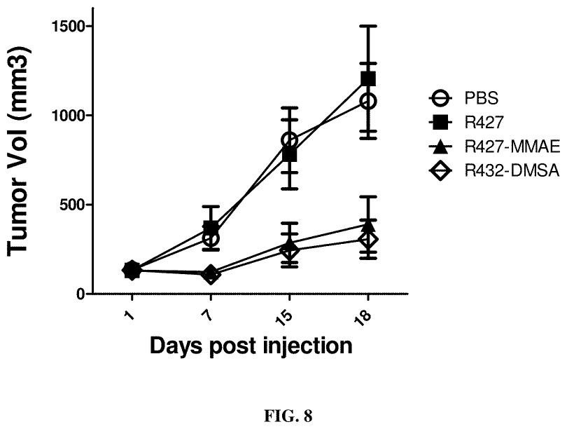

To evaluate the activity of drug conjugates of RSPO4-Fu-Q65R-Fc for anti-tumor activity in vivo, R427-MMAE and R432-DMSA were tested in xenograft models of LoVo cells. LoVo cells were implanted subcutaneously into the dorsal flanks of athymic nude mice, and when tumors reached the size of ˜150 mm³, the animals were randomized into 4 groups with 5-6 per group. The animals were injected with PBS (vehicle), unconjugated R427, R427-MMAE, or R432-DMSA at 5 mg/kg by intraperitoneal injection every other day for a total of seven injections. Tumor sizes were measured, and animals were monitored for general health. Both R427 and R432-DMSA showed strong anti-tumor effect (FIG. 8). No gross toxicity was observed with any of the treatment groups.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Barker et al., Development, 140:2484-2494, 2013.
Beck et al., Nature Rev., Drug Discov., 16:315-337, 2017.
Carmon et al., Proc Natl Acad Sci USA, 108:11452-11457, 2011.
Carmon et al. Mol Cell Biol., 32:2054-2064, 2012.
Chartier et al., Cancer Res, 76:713-723, 2016.
de Lau et al., Genome Biol., 13:242, 2012.
de Lau et al., Nature, 476:293-297, 2011.
de Sousa e Melo et al., Nature, 543:676-680, 2017.
Gong et al., Mol Cancer Ther, 15:1580-1590, 2016.
Gong et al., Oncogene, 34:4692-4701, 2015.
Junttila et al., Sci. Transl. Med., 7:314ra186, 2015.
Panowksi et al., MAbs, 6:34-45, 2014.
Park et al., J Biol Chem, 293:9759-9769, 2018.
Seshagiri et al., Nature, 488:660-664, 2012.
Shimokawa et al., Nature, 545:187-192, 2017.
Strop et al., Chem. Biol., 20:161-167, 2013.
Teicher, Curr. Cancer Drug Targets, 9:982-1004, 2009.
Yi et al., PLoS One, 8:e78144, 2013.

1. A polypeptide comprising a R-spondin (RSPO) domain that comprises a leucine-rich repeat containing, G protein-coupled receptor (LGR) binding sequence and a conjugated therapeutic agent.

2. The polypeptide of claim 1, wherein the polypeptide exhibits reduced Wnt signaling relative to a wild-type RSPO.

3. The polypeptide of claim 1, wherein the polypeptide exhibits reduced RNF43/ZNRF3 binding relative to a wild-type RSPO.

4. The polypeptide of claim 1, wherein the RSPO domain comprises a RSPO Fu1 domain and/or a RSPO TSP domain.

5. The polypeptide of claim 4, wherein the polypeptide comprises a mutation in the Fu1 domain relative to a wildtype RSPO, such as an amino acid substitution at a position corresponding to position Arg60, Gln65, and/or Gly67 of the RSPO domain (as numbered in an RSPO consensus sequence of FIG. 1).

6-8. (canceled)

9. The polypeptide of claim 1, wherein the RSPO domain comprises at least 85% identity to SEQ ID NOs: 1-4.

10-13. (canceled)

14. The polypeptide of claim 1, wherein the polypeptide further comprises a fused Ig Fc domain.

15. The polypeptide of claim 14, wherein the Ig Fc domain is an IgG Fc domain.

16. (canceled)

17. The polypeptide of claim 14, wherein the Ig Fc domain position C-terminal relative to the RSPO domain.

18-20. (canceled)

21. The polypeptide of claim 14, wherein the conjugated therapeutic agent is conjugated to the Fc domain.

22. The polypeptide of claim 14, further comprising an additional fused amino acid sequence and wherein the conjugated therapeutic agent is conjugated to the additional fused amino acid sequence.

23-25. (canceled)

26. The polypeptide of claim 1, wherein the conjugated therapeutic agent is conjugated via a PEG linker.

27. (canceled)

28. The polypeptide of claim 1, wherein the conjugated therapeutic agent is conjugated by a transglutaminase reaction.

29. The polypeptide of claim 1, wherein the polypeptide is at least 85% identical to SEQ ID NOs: 5-8.

30. The polypeptide of claim 1, wherein the conjugated therapeutic agent is cytotoxic agent, a chemotherapeutic agent, a radioactive isotope, an MMAE (monomethyl-auristatin) or DMSA.

31-35. (canceled)

36. A method of treating a subject comprising administering an effective amount of a polypeptide according to claim 1.

37-42. (canceled)

43. A polypeptide comprising a R-spondin (RSPO) domain that comprises a leucine-rich repeat containing, G protein-coupled receptor (LGR) binding sequence and fused Ig Fc domain.

44-77. (canceled)

78. A method of treating a subject comprising administering an effective amount of a polypeptide according to claim 43.

79-85. (canceled)
